(19) United States
(12) Patent Application Publication
(10) Pub. No.: US 2006/0206356 A1
(43) Pub. Date: Sep. 14, 2006

(54) Line verification for multi-pump arrays

(76) Inventor: Timothy W. Vanderveen, Poway, CA (US)

Correspondence Address: FULWIDER PATTON, 6060 CENTER DRIVE, 10TH FLOOR, LOS ANGELES, CA, 90045, US

(21) Appl. No.: 11/077,369

(22) Filed: Mar. 9, 2005

(51) Int. Cl. G06Q 10/00; A61M 29/00

(52) U.S. Cl. 705/2; 604/103.1

(57) ABSTRACT

A system and method for verifying that a medication delivery device is properly programmed to deliver a medication from a medication source. The system and method insure that the right medication is delivered in the right manner to a patient, and is particularly advantageous where multiple medications are being delivered simultaneously to a patient. The system and method also provides for ensuring that the device is properly programmed each time a medication to be delivered is changed by requiring verification each time an infusion pump is opened. The identification devices contemplated by the system includes barcodes affixed to the delivery device and medication. Other devices besides barcodes, such as RFID devices may also be used.

### FIELD OF THE INVENTION

The invention is directed to infusion pumps, and more specifically, arrays of infusion pumps, including multi-channel infusion pumps, that are configured to deliver more than one infusion fluid into a patient through multiple infusion lines.

### BACKGROUND OF THE INVENTION

In the environment of intensive care units, cardiac care units, operating rooms or trauma centers, it is often necessary to infuse into the patient more than one different drugs simultaneously. In addition, some of the drugs used in these environments are not directly compatible with each other and therefore need to be infused into the patient at different points of the body. Accordingly, arrays of infusion pumps are used to infuse each of the medications through different infusion lines. More recently, multi-channel infusion pumps capable of infusing several medications simultaneously at similar or different rates into a patient have been developed. While some types of these pumps are designed to deliver the medications through a common cannula, others are designed with multiple pumps, or channels, that pump fluid into a patient through a plurality of infusion lines.

As the name implies, multi-channel infusion pumps have more than one pumping channel, and an infusion line or set is installed into each channel. This arrangement allows each pump to be programmed to deliver the particular medication that flows through the infusion line or set installed in the channel such that each line may deliver mediation at different rates or in different volumes.

One problem that exists when infusing a patient with multiple infusion medications being delivered through different infusion lines is that it is necessary to ensure that each pump, or channel of a multi-channel pump, is properly programmed to deliver each medication. Since many infusion fluids and their sources look similar, one potential error is to load a drug into the wrong pump or pump channel. For example, drug A and infusion set A intended to be installed in pump A are mistakenly placed into pump B. Moreover, even where the proper drug and infusion set are initially installed in pump A, if the infusion fluid container must be changed during infusion, there is potential for the drug and infusion set to be placed in the wrong pump or channel at the time of replacement. Another potential error that can occur is that a pump or pump channel may be correctly programmed to deliver a particular medication, but the infusion line installed in the pump or pump channel is connected to the wrong medication source.

What has been needed, and not previously available, is a system and method of identifying infusion lines that ensures that the right line is installed in the right pumping channel. Such a system and method would include an automatic determination by the pumping channel that the right infusion line was installed. Alternatively, such a system could require some action on the part of the care giver before the pump would allow the infusion to begin.

### SUMMARY OF THE INVENTION

In one aspect, the present invention provides a method for matching an infusion channel with a medication source, comprising reading a barcode including information associated with an identification of a patient, reading a barcode including information associated with an identification of a medication to be delivered to the patient, comparing the information associated with the identification of the patient to the information associated with the identification of the medication, providing an alert signal if the comparison does not satisfy a predetermined condition, reading a barcode associated with a medication delivery device, the barcode including information associated with an identification of the medication delivery device, controlling a processor to accept operating parameters associated with the medication when the controller receives the information associated with the identification of the medication delivery device, and programming the medication delivery device to deliver the medication in accordance with the accepted operating parameters.

In another aspect of the present invention, programming the medication delivery device includes programming a selected one of a plurality of medication delivery devices, the selected one selected in accordance with the information associated with the identification of the medication delivery device.

In yet another aspect, the present invention provides a method from verifying that a medication delivery device selected from among a plurality of medication delivery devices is programmed to deliver a selected medication, comprising opening a door on the medication delivery device, installing an infusion line into the medication delivery device in a manner that engages the infusion line to cause an infusion fluid within the infusion line to be delivered to a patient, reading a barcode affixed to a surface of the medication delivery device that is accessible when the door of the medication delivery device is opened, the barcode including information representing an identity of the medication delivery device, reading a barcode affixed to a medication source in communication with the infusion line installed in the medication delivery device, the barcode including information representing an identity of the medication to be delivered to the patient, associating the medication delivery device with the medication source, and providing values of operating parameters associated with the medication to be delivered to the medication delivery device to operate the medication delivery device to deliver the medication to the patient.

In a still further aspect, the method of the present invention includes determining if more than one door of any of the plurality of medication delivery devices are open, and providing an alert that more than one door is open. In yet another aspect, the method of the present invention also includes detecting a door opening after initiating delivery of the medication, providing an alert that a door has been opened, and requiring that the barcode affixed to the medication delivery device and the barcode affixed to the medication source be read again before medication delivery may be restarted.

In yet another aspect, the method of the present invention includes a system for associating a medical delivery device with a medication source; comprising a medication delivery device, a medication delivery device identifier disposed on the medication delivery device, the medication delivery device identifier including information providing an identification of the medication delivery device, a medication source, a medication source identifier disposed on the medication source, the medication source identifier including information providing an identification of medication contained in the medication source, an identifier reader capable of reading the medication delivery device identifier and the medication source identifier and retrieving the identification information included in each of the medication delivery device identifier and the medication source identifier, input means for inputting values of device operating parameters in operable communication with the medication delivery device, and a processor in operable communication with the identifier reader and the medication deliver device, the processor programmed to receive the retrieved identification information, associate the medication source with the medication delivery device, receive the values of device operating parameters from the input means, communicate the values of device operating parameters to the medication delivery device and operate the medication deliver device in accordance with the values of device operating parameters.

In another aspect, the medication source identifier is a barcode, and in still another aspect, the medication source identifier is an RFID device. In yet another aspect, the medication delivery device identifier is a barcode, and in another aspect, the medication delivery device identifier is an RFID device.

Other features and advantages of the invention will become apparent from the following detailed description, taken in conjunction with the accompanying drawings, which illustrate, by way of example, the features of the invention.

### DESCRIPTION OF THE DRAWINGS

FIG. 1 is a schematic diagram of a patient care management system according to the present invention.

FIG. 2 is a graphic representation of a patient identification bracelet including a barcode that can be read by a barcode reader.

FIG. 3 is a drawing of a barcode label affixed to a medication container that can be read by a barcode reader.

FIG. 3A is a drawing of a barcode label affixed to a caregiver identity badge.

FIG. 4 is a drawing showing a sheet of barcode labels that can be affixed to various containers or devices.

FIG. 5 is a schematic diagram of a multi-channel pumping arrangement showing the pumping channels and multiple infusion sources and infusion lines.

FIG. 6 is a schematic diagram of a pump or pump channel showing a barcode label affixed to the pump housing adjacent a peristaltic infusion pump and infusion line.

### DESCRIPTION OF THE PREFERRED EMBODIMENTS

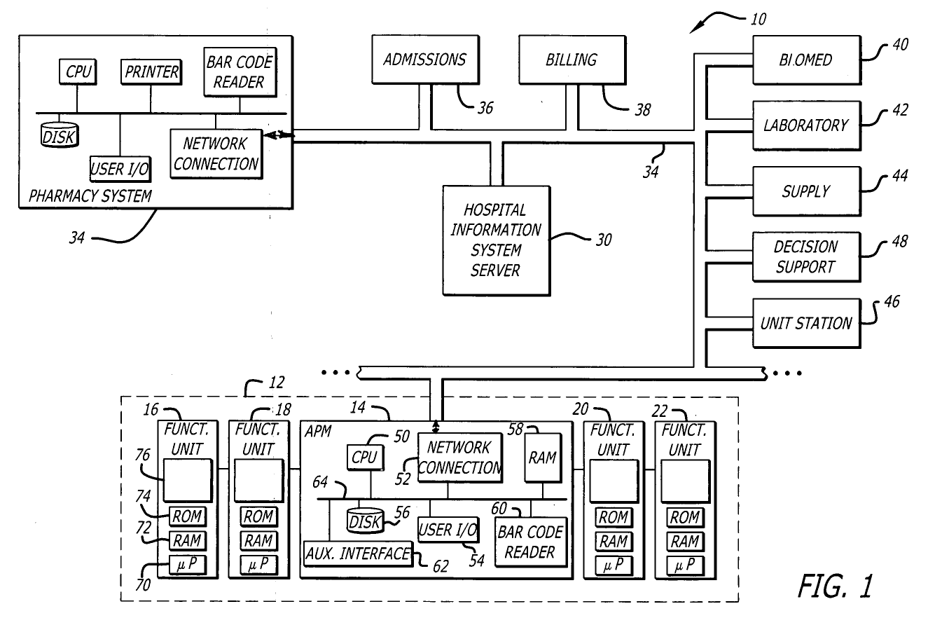

Referring now to the drawings in detail, in which like reference numerals indicate like or corresponding elements among the several figures, there is shown in FIG. 1 a general illustration of a patient care system in accordance with one embodiment of the present invention. In FIG. 1, a patient care device 12 is connected to a hospital network 10 including a pharmacy management system 34 and a hospital information system server 30. Each element 12, 30 and 34 is connected to network 10 by a transmission channel 32. Transmission channel 32 is any wired or wireless transmission channel, for example a 802.11 wireless local area network (LAN). In a preferred embodiment of the present invention, network 10 also includes computer systems located in various departments throughout a hospital. For example, network 10 of FIG. 1 optionally includes computer systems associated with an admissions department 36, a billing department 38, a biomedical engineering department 40, a clinical laboratory 42, a central supply department 44, one or more unit station computers and/or a medical decision support system 48.

Patient care device 12 preferably comprises a system similar to that described in U.S. Pat. No. 5,713,856 to Eggers et al., which is incorporated herein by reference. Alternatively, other patient care devices, such as pumps, physiological monitors (e.g., heart rate, blood pressure, ECG, EEG, pulse oximeter, and other patient monitors), therapy devices, and other drug delivery devices may be utilized according to the teachings set forth herein. Patient care device 12 preferably comprises an advanced programming module 14, also referred to as interface unit 14, connected to one or more functional modules 16, 18, 20, 22. Interface unit 14 includes a central processing unit (CPU) 50 connected to a memory, e.g. random access memory (RAM) 58, and one or more interface devices such as user interface device 54, a coded data input device 60, a network connection 52, and an auxiliary interface 62 for communicating with additional modules or devices. Interface unit 14 also preferably, although not necessarily, includes a main non-volatile storage unit 56, preferably a hard disk drive, for storing software and data and one or more internal buses 64 for interconnecting the aforementioned elements.

In a typical embodiment, user interface device 54 is a touch screen for displaying information to a user and allowing a user to input information by touching defined areas of the screen. Alternatively, user interface device 54 could include any means for displaying and inputting information, such as a monitor, a printer, a keyboard, softkeys, a mouse, a track ball and/or a light pen. Coded data input device 60 is preferably a bar code reader capable of scanning and interpreting data printed in bar coded format. Alternatively, data input device 60 could be any device for entering coded data into a computer, such as devices for reading a magnetic strips, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and coded data input device 60 may be the same device. Alternatively, although data input device 60 is shown in FIG. 1 to be disposed within interface unit 14, one skilled in the art will recognize that data input device 60 may be integral within pharmacy system 34 or located externally and communicating with pharmacy system 34 through an RS-232 serial interface or any other appropriate communication means. Auxiliary interface 62 is preferably an RS-232 communications interface, however any other means for communicating with a peripheral device such as a printer, patient monitor, infusion pump or other medical device may be used without departing from the scope of the invention.

Network connection 52 is preferably a direct network connection such as a TI connection, an integrated services digital network (ISDN) connection, a digital subscriber line (DSL) modem or a cable modem. Alternatively, any direct or indirect network connection may be used, including, but not limited to a telephone modem, an MIB system, an RS232 interface, an auxiliary interface, an optical link, an infrared link, a radio frequency link, a microwave link or a WLANS connection.

Functional modules 16, 18, 20, 22 are any devices for providing care to a patient or for monitoring patient condition. In preferred embodiment of the present invention, at least one of functional modules 16, 18, 20, 22 is an infusion pump module such as an intravenous infusion pump for delivering medication or other fluid to a patient. For the purposes of this discussion, functional module 16 is an infusion pump module. Each of functional modules 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor. Alternatively, functional module 18, 20 and/or 22 may be a printer, scanner or any other peripheral input/output device.

Each functional module 16, 18, 20, 22 communicates directly or indirectly with interface unit 14, with interface unit 14 providing overall monitoring and control of device 12. In a preferred embodiment, functional modules 16, 18, 20, 22 are connected physically and electronically in serial fashion to one or both ends of interface unit 14 as shown in FIG. 1 and as detailed in Eggers et al. However, one skilled in the art will recognize that there are other means for connecting functional modules with the interface unit may be utilized without departing from the scope of the invention. It will also be appreciated that devices such as pumps or monitors that provide sufficient programmability and connectivity may communicate directly with the network without a separate interface unit. As described above, additional medical devices or peripheral devices may be connected to patient care device 12 through one or more auxiliary interfaces 62.

Each functional module 16, 18, 20, 22 typically includes module-specific components 76, a microprocessor 70, a volatile memory 72 and a nonvolatile memory 74 for storing information. It should be noted that while four functional modules are shown in FIG. 1, any number of devices may be connected directly or indirectly to central computer 14. The number and type of functional modules described herein are intended to be illustrative, and in no way limit the scope of the present invention. Module-specific components 76 include any components necessary for operation of a particular module, such as a pumping mechanism for infusion pump module 16.

While each functional module is typically capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

In a preferred embodiment of the present invention, patient care device 12 is capable of operating in several different modes, or personalities, with each personality defined by a configuration database. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, e.g. from pharmacy 34, admissions 36, laboratory 42, etc.

Data to and from the various data sources can be converted into network-compatible data with existing technology, and movement of the information between the medical device and network can be accomplished by a variety of means. For example, patient care device 12 and network 10 may communicate via automated interaction and/or manual interaction. Automated interaction may be continuous or intermittent and may occur through direct network connection 54 (as shown in FIG. 1), or alternatively through RS232 links, MIB systems, RF links such as BLUETOOTH (Amtel Corp., San Jose, Calif.), IR links, WLANS, digital cable systems, telephone modems or other communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. Preferably, the communication means is bidirectional with access to data from as many points of the distributed data sources as possible. Decision-making can occur at a variety of places within network 10. For example, and not by way of limitation, decisions can be made in HIS server 30, decision support 48, hospital department or unit stations 46, or within patient care device 12 itself.

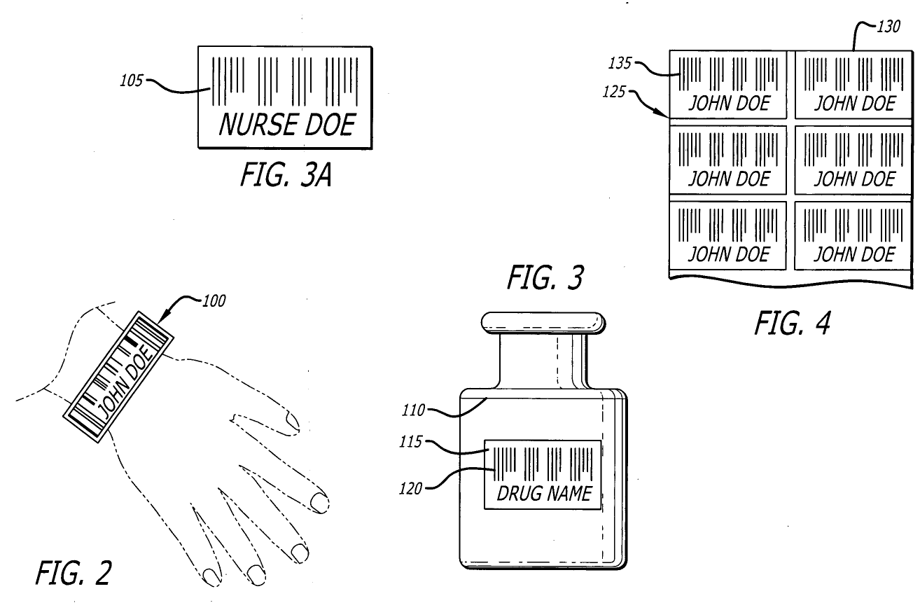

Referring now to FIGS. 2-4, a variety of implementations of the barcode identification system of the present invention are shown. FIG. 2, for example, shows a patient identification bracelet 100 of the kind typically used in hospitals and other institutional settings to ensure that each patient is able to be identified even if the patient is unconscious or otherwise unable to respond to questioning. A barcode 105 is printed on a label that is attached to the patient identification bracelet 100 and has encoded within its sequence of bars the information necessary to identify the patient. This barcode may be read using a computerized barcode reader, such as those identified by reference number 60 (FIG. 1). In one embodiment, the barcode reader 60 comprises a light emitting and receiving wand that is scanned across the barcode. The light emitted by the wand is reflected by the sequence of dark and light lines comprising the barcode into the receiving lens of the wand. A sensor in the wand converts the received light into a signal that is then transmitted to the CPU. A software application program running on the CPU then decodes the signal into the data represented by the barcode in a manner well known to one skilled in the art. Using appropriate software programs, this data may then be automatically entered into a database stored in the CPU's memory or disk storage. Alternatively, the sensor and suitable optics may be mounted within a module such that a badge or medication container is passed over a window that allows the emitted light to fall upon the bar code and be reflected back to the sensor.

Barcode systems are extremely flexible and the amount of information that can be represented by the barcode, while limited, can be used in a variety of ways. For example, as depicted in FIG. 3, a drug container 110 is identified by a label 115 having a barcode 120 printed thereon. This barcode 120 can represent the patient identification and the medical order number, and any other information the institution finds helpful in dispensing the drug and tracking the treatment, such as operating parameters from programming and controlling the medical device that is intended to deliver the medication. The barcode 120 may also be read using a barcode reader, and, using suitable application software can be used to link the drug container and its contents with the patient identification bracelet 100 affixed to a patient to ensure the right drug is delivered to the right patient at the right time in the right manner. The use of barcodes is not limited to the implementations discussed above. A sheet 190 of barcode labels 130 having barcodes 135 is shown in FIG. 4. Such labels can be printed by a printer connected to a pharmacy CPU or the care management system or, alternatively, by any other printer connected to any other hospital information system that can be programmed to produce barcodes bearing the information in a form that can be read by the barcode readers connected to the various CPUs of the care management system. These barcode labels 135 may then be affixed to clinical devices, patient belongings, or other items where positive identification is needed. As will be discussed in more detail below, barcodes may also be affixed to various clinical devices to assist in identifying the device. For example, a barcode may be placed on a pump and linked through appropriate software to the identification barcode of a medication container to ensure that the pump that will dispense a specific medication is programmed with operating parameters that are correct for the linked medication.

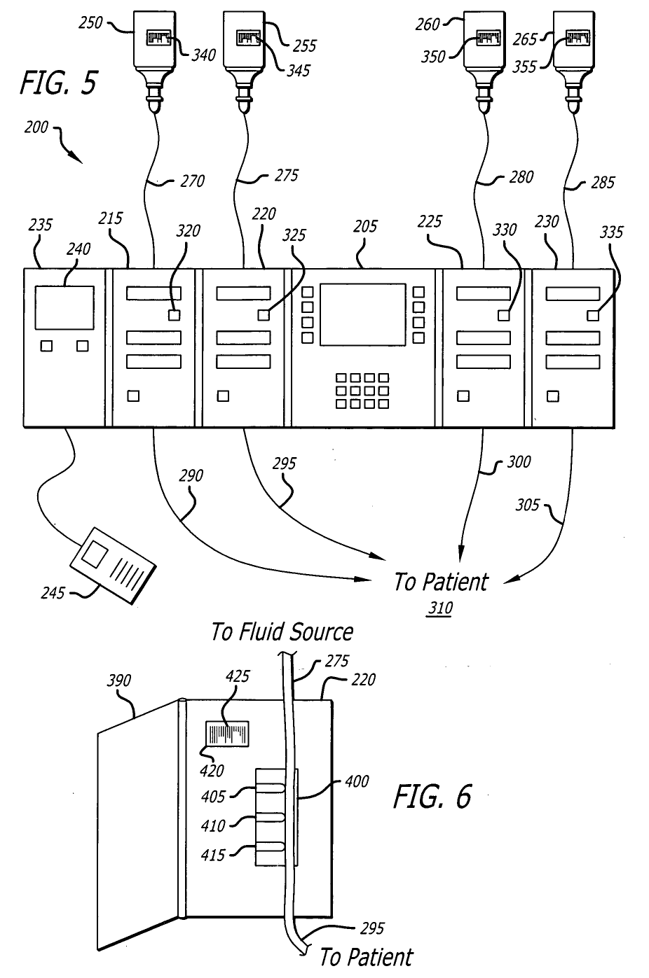

FIG. 5 depicts one embodiment of the present invention showing a setup 200 arranged to deliver multiple infusions to a patient. While FIG. 5 shows a multi-channel pumping arrangement, those skilled in the art will understand that such an arrangement could be replaced with an array of single, double or other configuration pumps and still accomplish the solution to the problem of ensuring that the right drugs are delivered by the right pumps at the right rates that is provided by the present invention.

The setup 200 depicted in FIG. 5 includes an interface unit/controller 205, four LVP infusion pumps 215, 220, 225 and 230, and a barcode module 235. Barcode module 235 includes a barcode reader 240 mounted within the barcode module 235, as well as a barcode reader wand 245 operable connected to the barcode module 235. The barcode reader wand 245 allows reading barcodes affixed to objects that cannot be placed directly in front of barcode reader 240.

As shown in FIG. 5, medication sources 250, 255, 260 and 265 containing medication to be delivered to a patient are connected to infusion lines 270, 275, 280 and 285 respectively. Each of these infusion lines is routed through one of infusion pumps 215, 220, 225 or 230. Downstream of each of the pumps is a continuation of the infusion line, here identified as 290, 295, 300 and 305, that communicates the medication to be delivered to the patient 310.

In the depicted embodiment, each of the pumps 215, 220, 225 and 230 have a barcode 320, 325, 330 and 335 affixed to the pump that may be read by the barcode wand 245 to identify the pump. Additionally, each medication source 250, 255, 260 and 265 has a barcode 340, 345, 350 and 355 fixed to it to identify the medication contained within the medication source. Moreover, each of barcodes 340, 345, 350 and 355 may contain additional information, such as operating information including values for various medication delivery parameters, such as volume to be infused, rate and the like that can be read by barcode wand 245 and used to program the processor of controller 205 to deliver the mediation contained within a specification medication source to the patient.

As is easily imagined, it is important that a medication delivery device such as infusion pumps 215, 220, 225 or 230 be properly programmed to ensure that the medication that each pump is delivering to the patient is delivered properly. One problem that exists in a busy institution where multiple infusions are being given to a single patient is the possibility that a care giver will make a mistake and program a pump for a medication other than the medication that is actually be delivered by the pump. Such errors arise because of the multitude of medication sources and lines can result in a confusing array of infusion lines that must be traced to a specific medication source. The care giver must then program the particular pump with the proper operating parameters.

The present invention solves this problem by providing a system and method for matching an infusion pump to a medication source. In one embodiment, the software operating on the controller 205 and pump use information provided by the barcode reader 235 to ensure that the proper pump or pumping channel is being programmed with operating parameters that are appropriate to the medication contained in the medication source connected to the pump. For example, in one embodiment, the caregiver scans an identification bracelet containing a barcode on a patient, and then scans the barcode 340 on medication source 250. The controller processes the information provided by the barcodes to see if the medication contained in medication source 250 is intended to be administered to that specific patient. If the controller determines that the right medication is being given to the right patient, the controller then allows, or in some embodiments, prompts, the care giver to read barcode 320 on pump 215.

When barcode 320 has been read, the controller assigns pump 215 to the medication contained in medication source 250, and then prompts the care giver to manually enter all information necessary to start the infusion, such as operating parameters such as infusion rate, volume and the like. By using the barcode reader 245 to identify the pump channel connected to medication source 250, the controller ensures that the delivery parameters associated with the medication contained in source 250 are programmed into the proper pump or pumping channel.

It will be understood that while the invention is described in terms of use of barcodes, other means for positively identifying patients, care givers, medications, and devices may be used, such as wireless devices. For example, each of the barcodes in to the above described example may be replaced with radio frequency identification devices (RFID). Such devices incorporate circuitry that is responsive to signals generated by a transmitter or transmitter/receiver to provide a transmission of information to the receiver. In such a system, the transmitter sends a query signal that is received by the RFID device. The RFID device responds to the signal by transmitting a signal containing identification information, or any other information it is programmed to transmit, that is then received by the receiver. Thus, the RFID device can provide the same sort of positive identification as the barcodes described above. Moreover, the system and method of the present invention contemplates the use of any combination of barcodes, RFID devices or other means for identification to ensure that the right drug is delivered to the right patient using the right delivery parameters.

FIG. 6 depicts a further embodiment of the present invention. In this embodiment, a barcode is mounted on an interior surface of pump 220, and can only be accessed by opening the door 390 of pump 220, such as when infusion line 275 is mounted in pumping portion 400 so that the line is in operable communication with pumping fingers 405, 410 and 415. In this embodiment, barcode label 420 having barcode 425 is read by the barcode reader 245. This provides further insurance that the right medication delivery parameters are being programmed into the right pump. In another embodiment, the software operating controller 205 may include routines that only allow the door of a single pump to be opened at any given time.

In a further scenario, controller 205 may require that barcode 425 be read and matched to a medication source barcode every time that the door 390 is opened. In this manner, controller 205 may insure that the pump is correctly programmed in the event that the door is opened to change an infusion line or for some other purpose, thus preventing inadvertent switching of lines from a source that would result in improper delivery of the medication contained in the source.

The comparisons required to carry out the present invention may be carried out in the processor of the controller 205 or the infusion pump 215. Additionally, since the controller 205 may be in communication with one or more internal or external servers associated with the institution, the comparisons may be carried out in any of the servers, with the appropriate commands and information being communicated between the server and the controller.

Additionally, the software instructions that are used to program the controller may also include capabilities such as providing for comparing values of operation parameters used to program and operate medication delivery devices to rules contained in a database of rules that have been established by the institution to ensure proper medication delivery. If any value of an operational value is not included within a range of predetermined acceptable values, such as set forth in a rule or rule as described above, the controller or server, depending on where the comparison takes place, may provide an alert to call attention to the discrepancy. Similarly, the rule may cause the system to pause until the discrepancy is corrected.

While several particular forms of the invention have been illustrated and described, it will be apparent that various modifications can be made without departing from the spirit and scope of the invention.

We claim:

1. A method for matching an infusion channel with a medication source, comprising:
reading a barcode including information associated with an identification of a patient;
reading a barcode including information associated with an identification of a medication to be delivered to the patient;
comparing the information associated with the identification of the patient to the information associated with the identification of the medication;
providing an alert signal if the comparison does not satisfy a predetermined condition;
reading a barcode associated with a medication delivery device, the barcode including information associated with an identification of the medication delivery device;
controlling a processor to accept operating parameters associated with the medication when the controller receives the information associated with the identification of the medication delivery device; and
programming the medication delivery device to deliver the medication in accordance with the accepted operating parameters.

2. The method of claim 1, wherein programming the medication delivery device includes programming a selected one of a plurality of medication delivery devices, the selected one selected in accordance with the information associated with the identification of the medication delivery device.

3. A method for verifying that a medication delivery device selected from among a plurality of medication delivery devices is programmed to deliver a selected medication, comprising:
opening a door on the medication delivery device;
installing an infusion line into the medication delivery device in a manner that engages the infusion line to cause an infusion fluid within the infusion line to be delivered to a patient;
reading a barcode affixed to a surface of the medication delivery device that is accessible when the door of the medication delivery device is opened, the barcode including information representing an identity of the medication delivery device;
reading a barcode affixed to a medication source in communication with the infusion line installed in the medication delivery device, the barcode including information representing an identity of the medication to be delivered to the patient;
associating the medication delivery device with the medication source; and
providing values of operating parameters associated with the medication to be delivered to the medication delivery device to operate the medication delivery device to deliver the medication to the patient.

4. The method of claim 3, further comprising:
determining if more than one door of any of the plurality of medication delivery devices are open; and
providing an alert that more than one door is open.

5. The method of claim 3, further comprising:
detecting a door opening after initiating delivery of the medication;
providing an alert that a door has been opened;
requiring that the barcode affixed to the medication delivery device and the barcode affixed to the medication source be read again before medication delivery may be restarted.

6. A system for associating a medical delivery device with a medication source; comprising:
a medication delivery device;
a medication delivery device identifier disposed on the medication delivery device, the medication delivery device identifier including information providing an identification of the medication delivery device;
a medication source;
a medication source identifier disposed on the medication source, the medication source identifier including information providing an identification of medication contained in the medication source;
an identifier reader capable of reading the medication delivery device identifier and the medication source identifier and retrieving the identification information included in each of the medication delivery device identifier and the medication source identifier;
input means for inputting values of device operating parameters in operable communication with the medication delivery device; and
a processor in operable communication with the identifier reader and the medication delivery device, the processor programmed to receive the retrieved identification information, associate the medication source with the medication delivery device, receive the values of device operating parameters from the input means, communicate the values of device operating parameters to the medication delivery device and operate the medication delivery device in accordance with the values of device operating parameters.

7. The system of claim 6, wherein the medication source identifier is a barcode.

8. The system of claim 6, wherein the medication source identifier is an RFID device.

9. The system of claim 6, wherein the medication delivery device identifier is a barcode.

10. The system of claim 6, wherein the medication delivery device identifier is an RFID device.
